(19) United States
(12) Patent Application Publication
(10) Pub. No.: US 2025/0377348 A1
(43) Pub. Date: Dec. 11, 2025

(54) TEST METHOD FOR DETERMINING DISPERSIVE SOIL THROUGH SOIL PASTE COLUMN WATER EROSION TEST

(71) Applicant: Northwest A&F University, Xi'an City (CN)

(72) Inventors: Henghui FAN, Xi'an City (CN); Xingyao LI, Xi'an City (CN); Guanzhou REN, Xi'an City (CN); Zengchun SUN, Xi'an City (CN); Ningrui XU, Xi'an City (CN); Jixiang WEN, Xi'an City (CN); Hongdong GUO, Xi'an City (CN); Feihan XIE, Xi'an City (CN); Baofeng LEI, Xi'an City (CN); Yuan GAO, Xi'an City (CN)

(21) Appl. No.: 18/740,277

(22) Filed: Jun. 11, 2024

(51) Int. Cl. G01N 33/24; G01N 1/28; G01N 1/38

(52) U.S. Cl. CPC G01N 33/24; G01N 1/286; G01N 1/38

(57) ABSTRACT

A test method for determining dispersive soil through soil paste column water erosion test is provided. A soil paste column water erosion test device comprising a water erosion tray, a fixing clamp, a rod frame, a water storage cylinder, a water stop clamp, a water conveying hose, a water outlet dripper, and a sample base is used. The water storage cylinder is fixed to an upper side of the rod frame with a rubber band, and is connected to the water outlet dripper through the water conveying hose. The water stop clamp is installed on the water conveying hose to control water flow pattern. The water outlet dripper is fixed by the fixing clamp located on the rod frame; the rod frame is fixed to the water erosion tray, and the water erosion tray is used for placing the sample base and a soil paste column sample.

### TECHNICAL FIELD

The present disclosure belongs to the technical field of geotechnical engineering test, and in particular to a test method for determining dispersive soil through soil paste column water erosion test.

### BACKGROUND

Dispersive soil is a special type of water-sensitive soil. The attraction between soil particles in low salt content water or purified water is less than the repulsion, and some soil particles will be dispersed into colloidal particles by themselves, resulting in poor resistance to water flow erosion, which easily causes damage such as internal piping and surface erosion of the soil, and is extremely harmful to civil engineering and water conservancy projects. The failure of projects such as the Wister Dam in Oklahoma, USA, some homogeneous dams in Western Australia, the San Juan Reservoir Dam in Spain, the Nenjiang River Diversion Project in Heilongjiang Province, and the Lingluo Reservoir in Hainan Province are all caused by the use of dispersive soil as filling material. At present, there are five test methods for determining the dispersibility of soil samples, such as pinhole test, crumb test, double densimeter test, pore water soluble cation test, and exchangeable sodium ion percentage test. The instruments and equipment involved in these test methods are complex, the test process is complicated, and the determination results are sometimes contradictory, so it is difficult to determine accurately. Meanwhile, due to high requirements for the environment, these test methods can not be carried out in field investigation, which brings a lot of inconveniences for quickly determining the dispersibility of soil.

### SUMMARY

To overcome the above shortcomings or disadvantages in the prior art, an objective of the present disclosure is to provide a test method for determining dispersive soil through soil paste column water erosion test, so as to rapidly and accurately determine the dispersibility of soil.

To achieve the above objective, the technical solution provided in the present disclosure is as follows:

A test method for determining dispersive soil through soil paste column water erosion test is provided. In the test method, a soil paste column water erosion test device comprising a water erosion tray, a fixing clamp, a rod frame, a water storage cylinder, a water stop clamp, a water conveying hose, a water outlet dripper, and a sample base is used for determining the dispersibility of soil. The water storage cylinder is fixed to an upper side of the rod frame with a rubber band, and is connected with the water outlet dripper through the water conveying hose. The water stop clamp is installed on the water conveying hose to control water flow pattern. The water outlet dripper is fixed by the fixing clamp located on the rod frame. The rod frame is fixed to the water erosion tray, and the water erosion tray is configured for placing the sample base and a soil paste column sample.

A method for specifically determining the dispersibility of soil includes the following steps:

(1) putting a proper amount of soil sample into a mixing bowl, adding water, and mixing the soil sample to a plastic state through a soil trowel for later use;

(2) putting the soil sample that has been mixed to the plastic state into a cutting ring, vibrating the cutting ring on a sample pad to exhaust air, and continuously adding the soil sample during vibration until the cutting ring is filled with the soil sample, so as to form a dense and uniform soil paste column sample;

(3) leveling a top of the soil paste column sample, pressing a molding cover into the top for a moment, a cylindrical ridge and a linear ridge are formed at an inner side of the molding cover, and removing the molding cover, such that a cylindrical groove and a linear groove are formed on the top of the soil paste column sample;

(4) pushing the soil paste column sample out of the cutting ring through a sample pusher;

(5) placing the soil paste column sample on a sample base, and placing the soil paste column sample and the sample base together into the water erosion tray, adjusting the water outlet dripper to keep a distance between the water outlet and a center of a top surface of the soil paste column sample fixed;

(6) adding water into the water storage cylinder, adjusting the water stop clamp to dropwise add the water into the center of the top surface of the soil paste column sample; and

(7) at last observing water erosion state of a side surface of the soil paste column sample and color characteristics of a water flow in the water erosion tray, so as to determine the dispersibility of the soil sample.

According to the present disclosure, in Step (1), a soil sample with natural moisture content is used, the soil sample is crushed and mixed evenly, and a sufficient amount of soil sample is taken by quartering method for late use, if the soil sample is of high moisture content, the soil sample should be dried before use.

In Step (3), when the molding cover is removed, edges of the cylinder groove and the linear groove formed in the top surface of the soil paste column sample should be clear and complete.

In Step (4), the sample pusher is placed on the sample pad, then the cutting ring is placed on the sample pusher, and the sample is slowly pushed out of the cutting ring. Disturbance should be avoided as much as possible during the operation to ensure that the side surface of the soil paste column sample is smooth.

In Step (5), the distance between the water outlet of the water outlet dripper and the center of the top surface of the soil paste column is fixed, so as to ensure that water drops are added into the cylindrical groove of the soil paste column sample.

In Step (6), the water drops should be added dropwise, rather than flowing in a form of streams or jets, into the cylindrical groove on the top surface of the soil paste column sample.

In Step (7), standard for determining the dispersibility of the soil sample is as follows:

water erosion for a non-dispersive soil paste column sample: water flows down from the pressed groove on a surface of the sample, no water erosion channel is formed on the side surface of the soil paste column sample, and the water flowing into the water erosion tray is clear;

water erosion for a transitional soil paste column sample: water flows down from the pressed groove on the surface of the sample, no or a few water erosion channels are formed on the side surface of the soil paste column sample, and the water flowing into the water erosion tray is slightly turbid; and

water erosion for a dispersive soil paste column sample: water flows down from the pressed groove on the surface of the sample, obvious water erosion channels are formed on the side surface of the soil paste column sample, and the water flowing into the water erosion tray is quite turbid.

Compared with the prior art, the test method for determining dispersive soil through soil paste column water erosion test has the following beneficial effects:

(1) The physical meaning of the test process is clear, the phenomenon of the test results is clear, and thus the dispersibility of the soil can be directly and accurately determined.

(2) The soil paste column water erosion test device has a simple structure and low requirements for test environment, and can be carried out in the field or indoors, thus the test cost is effectively reduced.

(3) The test method is convenient to operate, the entire test process is short, the test efficiency is greatly improved, and the reproducibility of the test results is good.

### BRIEF DESCRIPTION OF DRA WINGS

FIG. 1 is a schematic diagram of a soil paste column water erosion test device according

to an embodiment;

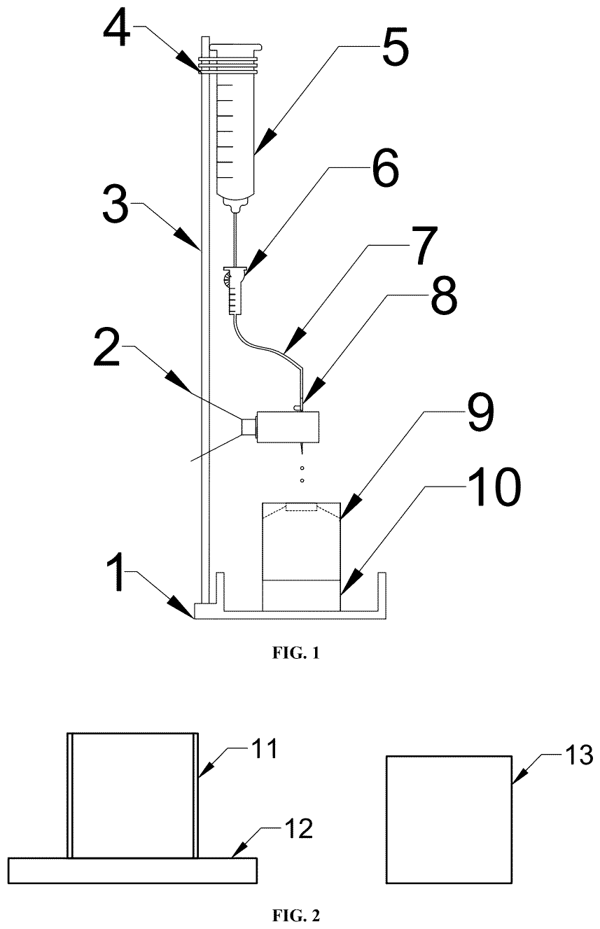

FIG. 2 is a schematic diagram of a sample preparation device;

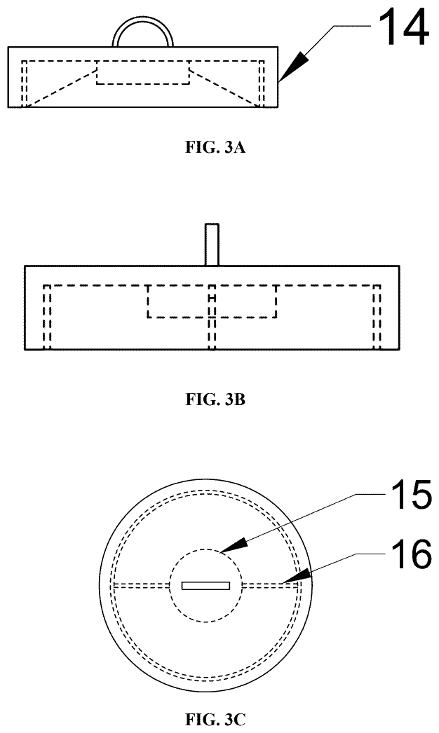

FIG. 3A is a front view of a molding cover; FIG. 3B is a side view of a molding cover;

FIG. 3C is a top view of a molding cover;

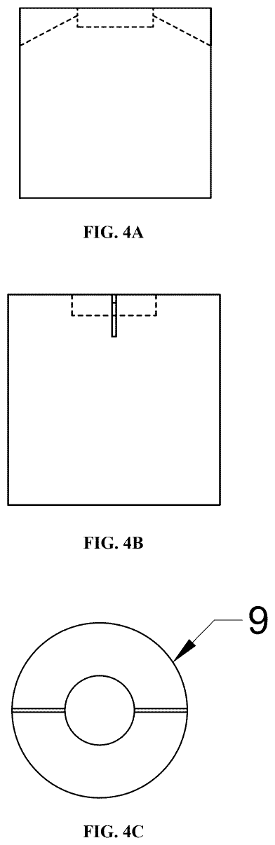

FIG. 4A is a front view of a soil paste column sample; FIG. 4B is a side view of a soil paste column sample; FIG. 4C is a top view of a soil paste column sample.

Reference signs in the drawings: 1, water erosion tray; 2, fixing clamp; 3, rod frame; 4, rubber band; 5, water storage cylinder; 6, water stop clamp; 7, water conveying hose; 8, water outlet dripper; 9, soil paste column sample; 10, sample base; 11, cutting ring; 12, sample pad; 13, sample pusher; 14, molding cover; 15, cylindrical ridge; 16, linear ridge.

The present disclosure is further described below in detail with reference to accompanying drawings and embodiments.

### DETAILED DESCRIPTION OF THE INVENTION

Referring to FIG. 1 to FIG. 4, a test method for determining the dispersibility of soil through soil paste column water erosion test according to this embodiment is provided. As shown in FIG. 1, the device comprises a water erosion tray 1, a fixing clamp 2, a rod frame 3, a rubber band 4, a water storage cylinder 5, a water stop clamp 6, a water conveying hose 7, a water outlet dripper 8, and a sample base 10. The water storage cylinder 5 is fixed to an upper side of the rod frame 3 with a rubber band 4, and is connected with the water outlet dripper 8 through the water conveying hose 7. The water stop clamp 6 is installed on the water conveying hose 7 to control water flow pattern. The water outlet dripper 8 is fixed by the fixing clamp 2 located on the rod frame 3. The rod frame 3 is fixed to the water erosion tray 1, and the water erosion tray 1 is configured for placing the sample base 10 and a soil paste column sample 9.

As shown in FIGS. 2, 3A, 3B, and 3C, a sample preparation device for the soil paste column sample comprises a cutting ring 11, a sample pad 12, a sample pusher 13, and a molding cover 14.

As shown in FIGS. 3A, 3B, and 3C, the molding cover is in the shape of a circular cover with a handle on an outer side and a protrusion on an inner side. Two kinds of protrusions are formed at the inner side of the molding cover 14: a cylindrical ridge 15, and a linear ridge 16. The prepared soil paste column sample is shown in FIGS. 4A, 4B, and 4C.

A method for specifically determining the dispersibility of soil includes the following steps:

(1) putting a proper amount of soil sample into a mixing bowl, adding water, and mixing the soil sample to a plastic state through a soil trowel for later use;

(2) putting the soil sample that has been mixed to the plastic state into the cutting ring 11, and vibrating the soil sample on the sample pad 12 to exhaust air, and continuously adding the soil sample during vibration until the cutting ring 11 is filled with the soil sample, so as to form a dense and uniform soil paste column sample;

(3) leveling a top of the soil paste column sample, pressing the molding cover 14 into the top for a moment, a cylindrical ridge and a linear ridge are formed at an inner side of the molding cover 14, and removing the molding cover 14, such that a cylindrical groove and a linear groove are formed on the top of the soil paste column sample 9;

(4) pushing the soil paste column sample 9 out of the cutting ring 11 through a sample pusher 13;

(5) placing the soil paste column sample 9 on a sample base 10, and placing the soil paste column sample and the sample base together into the water erosion tray 1, adjusting the water outlet dripper 8 to keep a distance between the water outlet and a center of a top surface of the soil paste column sample 9 fixed;

(6) adding water into the water storage cylinder 5, adjusting the water stop clamp 6 to dropwise add the water into the center of the top surface of the soil paste column sample; and

(7) at last observing water erosion state of a side surface of the soil paste column sample 9 and color characteristics of a water flow in the water erosion tray 1, so as to determine the dispersibility of the soil sample.

In this embodiment, in Step (1), a soil sample with natural moisture content is used, the soil sample is crushed and mixed evenly, and a sufficient amount of soil sample is taken by quartering method for late use. The sample with the natural moisture content should be used in this test. If the soil sample is of high moisture content, the soil sample may be properly dried before use.

In Step (3), when the molding cover 14 is removed, edges of the cylinder groove and the linear groove formed in the top surface of the soil paste column sample 9 should be clear and complete.

In Step (4), the sample pusher 13 is placed on the sample pad 12, then the cutting ring 11 is placed on the sample pusher 13, and the sample is slowly pushed out of the cutting ring 11. Disturbance should be avoided as much as possible during the operation to ensure that the side surface of the soil paste column sample is smooth.

In Step (5), the distance between the water outlet of the water outlet dripper 8 and the center of the top surface of the soil paste column sample 9 is fixed, so as to ensure that water drops are added into the cylindrical groove of the soil paste column sample 9.

In Step (6), the water drops should be added dropwise, rather than flowing in a form of streams or jets, into the cylindrical groove on the top surface of the soil paste column sample 9.

In Step (7), standard for determining the dispersibility of soil is shown as follows:

| Category | Water erosion characteristics of a soil paste column |
|---|---|
| Non-dispersive | water flows down from the pressed groove on a surface of the sample, no |
| soil | water erosion channel is formed on the side surface of the soil paste column |
| | sample, and the water flowing into the water erosion tray is clear. |
| Transitional soil | water flows down from the pressed groove on the surface of the sample, no or |
| | a few water erosion channels are formed on the side surface of the soil paste |
| | column sample, and the water flowing into the water erosion tray is slightly |
| | turbid. |
| Dispersive soil | water flows down from the pressed groove on the surface of the sample, |
| | obvious water erosion channels are formed on the side surface of the soil |
| | paste column sample, and the water flowing into the water erosion tray is |
| | quite turbid. |

The following is the specific embodiments provided by the inventor.

### EMBODIMENT 1

(1) 200 g of soil sample is put into a mixing bowl, a proper amount of pure water is added, and the soil sample is mixed to a plastic state through a soil trowel for later use.

(2) The mixed soil sample is put into a cutting ring 11, the cutting ring 11 is placed on a sample pad 12, and is vibrated to exhaust the air, the soil sample is continuously added during vibration until the cutting ring 11 is filled with the soil sample, so as to form a dense and uniform soil paste column sample.

In this embodiment, the cutting ring 11 has an inner diameter of 50 mm, and a height of 50 mm. The sample pad 12 has a diameter of 100 mm, and a thickness of 10 mm.

(3) The top of the soil paste column sample is leveled, and a molding cover 14 is pressed into soil of the soil paste column sample at the top of the cutting ring 11 for a moment, and the molding cover 14 is removed.

In this embodiment, the molding cover 14 is made of stainless steel, and a semicircular

handle is arranged at an upper end of the molding cover 14. Two kinds of protrusions are formed at an inner side of the molding cover 14: a cylindrical ridge 15, and a linear ridge 16. The cylindrical ridge 15 has a diameter of 20 mm and a height of 5 mm. The linear ridge 16 has a length of 15 mm, a thickness of 1 mm, an inner edge height of 2 mm, and an outer edge height of 10 mm.

(4) The soil paste column sample 9 is pushed out of the cutting ring 11 through the sample pusher 13. The pushed-out soil paste column sample 9 has a diameter of 50 mm, and a height of 50 mm, and a clear and complete cylindrical groove and a linear groove are formed at a top surface of the soil paste column sample.

In this embodiment, the sample pusher 13 has a diameter of 49 mm, and a height of 53 mm.

(5) The water erosion tray 1, the rod frame 3, the fixing clamp 2, the water storage cylinder 5, the water conveying hose 7, the water stop clamp 6, and the water outlet dripper 8 are assembled according to FIG. 1 to form a soil paste column water erosion test device. The water storage cylinder 5 is fixed to an upper side of the rod frame 3 with a rubber band 4, and is connected with the water outlet dripper 8 through the water conveying hose 7. The water stop clamp 6 is installed on the water conveying hose 7 to control water flow pattern. The water outlet dripper 8 is fixed by the fixing clamp 2 located on the rod frame 3. The rod frame 3 is fixed to the water erosion tray 1, and the water erosion tray 1 is configured for placing the sample base 10 and a soil paste column sample 9.

(6) The soil paste column sample 9 is placed on a sample base 10, and the soil paste column sample 9 and the sample base 10 together are placed into the water erosion tray 1, and a water outlet of the water outlet dripper 8 is adjusted, such that a distance between the water outlet and the center of the top surface of the soil paste column sample 9 is 10 cm.

In this embodiment, the sample base 10 has a diameter of 50 mm, and a height of 10 mm.

(7) 20 mL of pure water is added into the water storage cylinder 5, a drop rate of the pure water is adjusted to 40-60 drops/min by the water stop clamp 6, and the water drops are dropwise added into the circular groove on the top surface of the soil paste column sample 9.

(8) At last, water erosion state of the side surface of the soil paste column sample 9 and color characteristics of a water flow in the water erosion tray 1 are observed, it is found that no water erosion channel is formed on the side surface of the soil paste column sample 9, and the water flowing into the water erosion tray 1 is clear. Therefore, the soil sample belongs to non-dispersive soil.

### EMBODIMENT 2

(1) Another 200 g of soil sample is put into a mixing bowl, a proper amount of pure water is added, and the soil sample is mixed to a plastic state through a soil trowel for later use.

(2) The mixed soil sample is put into a cutting ring 11, the cutting ring 11 is placed on a sample pad 12, and is vibrated to exhaust the air, the soil sample is continuously added during vibration until the cutting ring 11 is filled with the soil sample, so as to form a dense and uniform soil paste column sample.

In this embodiment, the cutting ring 11 has an inner diameter of 50 mm, and a height of 50 mm. The sample pad 12 has a diameter of 100 mm, and a thickness of 10 mm.

(3) The top of the soil paste column sample is leveled, and a molding cover 14 is pressed into soil of the soil paste column sample at the top of the cutting ring 11 for a moment, and the molding cover 14 is removed.

In this embodiment, the molding cover 14 is made of stainless steel, and a semicircular handle is arranged an upper end of the molding cover 14. Two kinds of protrusions are formed at an inner side of the molding cover 14: a cylindrical ridge 15, and a linear ridge 16. The cylindrical ridge 15 has a diameter of 20 mm and a height of 5 mm. The linear ridge 16 has a length of 15 mm, a thickness of 1 mm, an inner edge height of 2 mm, and an outer edge height of 10 mm.

(4) The soil paste column sample 9 is pushed out of the cutting ring 11 through the sample pusher 13. The pushed-out soil paste column sample 9 has a diameter of 50 mm, and a height of 50 mm, and a clear and complete cylindrical groove and a linear groove are formed at a top surface of the soil paste column sample.

In this embodiment, the sample pusher 13 has a diameter of 49 mm, and a height of 53 mm.

(5) The water erosion tray 1, the rod frame 3, the fixing clamp 2, the water storage cylinder 5, the water conveying hose 7, the water stop clamp 6, and the water outlet dripper 8 are assembled according to FIG. 1 to form a soil paste column water erosion test device. The water storage cylinder 5 is fixed to an upper side of the rod frame 3 with a rubber band 4, and is connected with the water outlet dripper 8 through the water conveying hose 7. The water stop clamp 6 is installed on the water conveying hose 7 to control water flow pattern. The water outlet dripper 8 is fixed by the fixing clamp 2 located on the rod frame 3. The rod frame 3 is fixed to the water erosion tray 1, and the water erosion tray 1 is configured for placing of the sample base 10 and a soil paste column sample 9.

(6) The soil paste column sample 9 is placed on a sample base 10, and the soil paste column sample 9 and the sample base 10 together are placed into the water erosion tray 1, and a water outlet of the water outlet dripper 8 is adjusted, such that a distance between the water outlet and the center of the top surface of the soil paste column sample 9 is 10 cm.

In this embodiment, the sample base 10 has a diameter of 50 mm, and a height of 10 mm.

(7) 20 mL of pure water is added into the water storage cylinder 5, a drop rate of the pure water is adjusted to 40-60 drops/min by the water stop clamp 6, and the water drops are dropwise added into the circular groove on the top surface of the soil paste column sample 9.

(8) At last, water erosion state of the side surface of the soil paste column sample 9 and color characteristics of a water flow in the water erosion tray 1 are observed, it is found that a few water erosion channels are formed on the side surface of the soil paste column sample 9, and the water flowing into the water erosion tray 1 is slightly turbid. Therefore, the soil sample belongs to transitional soil.

### EMBODIMENT 3

(1) Still another 200 g of soil sample is put into a mixing bowl, a proper amount of pure water is added, and the soil sample is mixed to a plastic state through a soil trowel for later use.

(2) The mixed soil sample is put into a cutting ring 11, the cutting ring 11 is placed on a sample pad 12, and is vibrated to exhaust the air, the soil sample is continuously added during vibration until the cutting ring 11 is filled with the soil sample, so as to form a dense and uniform soil paste column sample.

In this embodiment, the cutting ring 11 has an inner diameter of 50 mm, and a height of 50 mm. The sample pad 12 has a diameter of 100 mm, and a thickness of 10 mm.

(3) The top of the soil paste column sample is leveled, and a molding cover 14 is pressed into soil of the soil paste column sample at the top of the cutting ring 11 for a moment, and the molding cover 14 is removed.

In this embodiment, the molding cover 14 is made of stainless steel, and a semicircular handle is arranged at an upper end of the molding cover 14. Two kinds of protrusions are formed at an inner side of the molding cover 14: a cylindrical ridge 15, and a linear ridge 16. The cylindrical ridge 15 has a diameter of 20 mm and a height of 5 mm. The linear ridge 16 has a length of 15 mm, a thickness of 1 mm, an inner edge height of 2 mm, and an outer edge height of 10 mm.

(4) The soil paste column sample 9 is pushed out of the cutting ring 11 through the sample pusher 13. The pushed-out soil paste column sample 9 has a diameter of 50 mm, and a height of 50 mm, and a clear and complete cylindrical groove and a linear groove are formed at a top surface of the soil paste column sample.

In this embodiment, the sample pusher 13 has a diameter of 49 mm, and a height of 53 mm.

(5) The water erosion tray 1, the rod frame 3, the fixing clamp 2, the water storage cylinder 5, the water conveying hose 7, the water stop clamp 6, and the water outlet dripper 8 are assembled according to FIG. 1 to form a soil paste column water erosion test device. The water storage cylinder 5 is fixed to an upper side of the rod frame 3 with a rubber band 4, and is connected with the water outlet dripper 8 through the water conveying hose 7. The water stop clamp 6 is installed on the water conveying hose 7 to control water flow pattern. The water outlet dripper 8 is fixed by the fixing clamp 2 located on the rod frame 3. The rod frame 3 is fixed to the water erosion tray 1, and the water erosion tray 1 is configured for placing the sample base 10 and a soil paste column sample 9.

(6) The soil paste column sample 9 is placed on a sample base 10, and the soil paste column sample 9 and the sample base 10 together are placed into the water erosion tray 1, and a water outlet of the water outlet dripper 8 is adjusted, such that a distance between the water outlet and the center of the top surface of the soil paste column sample 9 is 10 cm.

In this embodiment, the sample base 10 has a diameter of 50 mm, and a height of 10 mm.

(7) 20 mL of pure water is added into the water storage cylinder 5, a drop rate of the pure water is adjusted to 40-60 drops/min by the water stop clamp 6, and the water drops are dropwise added into the circular groove on the top surface of the soil paste column sample 9.

(8) At last, water erosion state of the side surface of the soil paste column sample 9 and color characteristics of a water flow in the water erosion tray 1 are observed, it is found that obvious water erosion channels are formed on the side surface of the soil paste column sample 9, and the water flowing into the water erosion tray 1 is quite turbid. Therefore, the soil sample belongs to dispersive soil.

It should be noted that above embodiments are only the preferred embodiments of the present disclosure and the present disclosure is not limited to the above embodiments. It should be understood by those skilled in the art that the details and forms of the technical solution of the present disclosure can be modified or replaced without departing from the spirit and scope of the present disclosure and these modifications and replacements belong to the scope of protection defined by the claims of the present disclosure.

What is claimed is:

1. A test method for determining dispersive soil through soil paste column water erosion test, wherein a soil paste column water erosion test device comprising a water erosion tray, a fixing clamp, a rod frame, a water storage cylinder, a water stop clamp, a water conveying hose, a water outlet dripper, and a sample base is configured for determining the dispersibility of soil; wherein the water storage cylinder is fixed to an upper side of the rod frame with a rubber band, and is connected with the water outlet dripper through the water conveying hose; the water stop clamp is installed on the water conveying hose to control water flow pattern; the water outlet dripper is fixed by the fixing clamp located on the rod frame; the rod frame is fixed to the water erosion tray, and the water erosion tray is configured for placing of the sample base and a soil paste column sample;
wherein the test method for determining dispersibility of soil comprises the following steps:
(1) putting a proper amount of soil sample into a mixing bowl, adding water, and mixing the soil sample to a plastic state through a soil trowel for later use;
(2) putting the soil sample that has been mixed to the plastic state into a cutting ring, vibrating the cutting ring on a sample pad to exhaust air, and continuously adding the soil sample during vibration until the cutting ring is filled with the soil sample, so as to form a dense and uniform soil paste column sample;
(3) leveling a top of the soil paste column sample, pressing a molding cover into the top for a moment, wherein a cylindrical ridge and a linear ridge are formed at an inner side of the molding cover, and removing the molding cover, such that a cylindrical groove and a linear groove are formed on the top of the soil paste column sample;
(4) pushing the soil paste column sample out of the cutting ring through a sample pusher;
(5) placing the soil paste column sample on a sample base, and placing the soil paste column sample and the sample base together into the water erosion tray, adjusting the water outlet dripper to keep a distance between the water outlet and a center of a top surface of the soil paste column sample fixed;
(6) adding water into the water storage cylinder, adjusting the water stop clamp to dropwise add the water into the center of the top surface of the soil paste column sample; and
(7) At last, observing a water erosion state of a side surface of the soil paste column sample and color characteristics of a water flow in the water erosion tray, so as to determine the dispersibility of the soil sample.

2. The method according to claim 1, wherein in Step (1), a soil sample with natural moisture content is used, the soil sample is crushed and mixed evenly, and a sufficient amount of soil sample is taken by quartering method for late use, if the soil sample is of high moisture content, the soil sample should be dried before use.

3. The method according to claim 1, wherein in Step (3), when the molding cover is removed, edges of the cylinder groove and the linear groove formed in the top surface of the soil paste column sample are clear and complete.

4. The method according to claim 1, wherein in Step (4), the cutting ring is placed on the sample pusher, and the sample is slowly pushed out of the cutting ring; and disturbance should be avoided as much as possible during the operation to ensure that the side surface of the soil paste column sample is smooth.

5. The method according to claim 1, wherein in Step (5), the distance between the water outlet of the water outlet dripper and the center of the top surface of the soil paste column is fixed, so as to ensure that water drops are added into the cylindrical groove of the soil paste column sample.

6. The method according to claim 1, wherein in Step (6), the water drops should be added dropwise, rather than flowing in a form of streams or jets, into the cylindrical groove on the top surface of the soil paste column sample.

7. The method according to claim 1, wherein in Step (7), standard for determining the dispersibility of the soil sample is as follows:
water erosion for a non-dispersive soil paste column sample: water flows down from the pressed groove on a surface of the sample, no water erosion channel is formed on the side surface of the soil paste column sample, and the water flowing into the water erosion tray is clear;
water erosion for a transitional soil paste column sample: water flows down from the pressed groove on the surface of the sample, no or a few water erosion channels are formed on the side surface of the soil paste column sample, and the water flowing into the water erosion tray is slightly turbid; and
water erosion for a dispersive soil paste column sample: water flows down from the pressed groove on the surface of the sample, obviously water erosion channels are formed on the side surface of the soil paste column sample, and the water flowing into the water erosion tray is quite turbid.
